(19) United States
(12) Patent Application Publication
(10) Pub. No.: US 2026/0251628 A1
(43) Pub. Date: Aug. 27, 2026

(54) UPPER ATMOSPHERIC ENVIRONMENTAL GAS MEASUREMENT DEVICE FOR RADIOSONDE AND MEASUREMENT METHOD THEREOF

(71) Applicant: Korea Research Institute of Standard and Science, Daejeon (KR)

(72) Inventors: Yong-Gyoo Kim, Daejeon (KR); Sang Wook Lee, Daejeon (KR); Seon Woong Kim, Daejeon (KR); Sunghun Kim, Daejeon (KR); Youngseok Lee, Daejeon (KR); Jae-keun Yoo, Daejeon (KR)

(73) Assignee: Korea Research Institute of Standard and Science, Daejeon (KR)

(21) Appl. No.: 19/292,099

(22) Filed: Aug. 6, 2025

(30) Foreign Application Priority Data
Feb. 25, 2025 (KR) 10-2025-0024403

(51) Int. Cl. G01N 33/00; G01K 13/024; G01N 1/22; G01W 1/08

(52) U.S. Cl. CPC G01N 33/0016; G01K 13/024; G01N 1/2273; G01N 33/0031; G01N 33/0037; G01N 33/0039; G01N 33/0042; G01W 1/08; G01N 2001/2279

(57) ABSTRACT

The present disclosure relates to a compact, lightweight, and inexpensively manufactured upper atmospheric environmental gas measurement device for a radiosonde, and a measurement method thereof. To this end, an upper atmospheric environmental gas measurement device (100) for a radiosonde (300), includes an insulation pack having an internal space (160), an air intake port (170) whose one end is exposed to an outside of the insulation pack to allow external air to flow in and whose other end extends to the internal space (160), a heater (176) connected to the other end of the air intake port (170) to heat the external air to room temperature, an environmental gas sensor (190) for measuring a concentration of an environmental gas contained in the heated external air, a temperature sensor (210) for measuring temperature of the external air passing through the environmental gas sensor (190), a pump inlet pipe (220) whose one end is connected to the environmental gas sensor (190) and whose middle region passes through the temperature sensor (210), an air pump (230) connected to the other end of the pump inlet pipe (220) and having an outlet (235) exposed to the internal space (160), a control unit (200) controlling the heater (176) based on output of the temperature sensor (210), an X-data communication module (260) transmitting an output signal of the environmental gas sensor (190) to the outside, and a battery (180) supplying power to the upper atmospheric environmental gas measurement device (100).

### CROSS-REFERENCE TO RELATED APPLICATIONS

This application claims the priority of Korean Patent Application No. 10-2025-0024403 filed on Feb. 25, 2025, in the Korean Intellectual Property Office, the disclosure of which is incorporated herein by reference.

### BACKGROUND

### Field

The present disclosure relates to a gas measurement device for a radiosonde, and more specifically, to a small, lightweight, and inexpensively manufactured upper atmospheric environmental gas measurement device for a radiosonde and a measurement method thereof.

### Description of the Related Art

To monitor environmental gases such as nitrogen dioxide (NO₂) and ozone (O₃), measurements are made periodically using airplanes at low altitudes below 10 km. In addition, to measure ozone in the high-altitude stratosphere, specialized equipment that is expensive, complex, and not accessible for routine or widespread use is required.

These conventional measurement systems are bulky and expensive, often requiring dedicated payloads on research aircraft, high-altitude balloons, or satellite missions. For example, in the case of upper atmosphere environmental gas measurements, instruments used for Differential Optical Absorption Spectroscopy (DOAS) are highly sensitive, but cost more than $50,000 and require significant power and data processing capabilities.

Moreover, although ozone sensors using electrochemical concentration cells (ECCs) are used in some radiosondes, they require regular calibration and have very limited capacity for measuring multiple gases simultaneously. In addition, there is a disadvantage that the ozone sensors are expensive, heavy, and have complex preprocessing processes before launching. Nevertheless, actually, the ozone sensors are the most widely used methods in the field.

Moreover, Cavity Ring-Down Spectroscopy (CRDS) is a state-of-the-art technology for gas analysis, but it is a very expensive piece of equipment, costing over $100,000, and is only used in some labs or high-budget field research projects.

A tropospheric monitoring instrument (TROPOMI) has global applicability but are expensive to deploy, inaccessible for regional or local studies, and lack the fine vertical resolution required for atmospheric profiling.

Reliance on such expensive and resource-intensive conventional equipment constrains the ability to perform high-resolution atmospheric gas measurements frequently in developing countries and in scenarios where rapid deployment and economic feasibility are required. These constraints hinder comprehensive monitoring of air pollutants and impede the ability to address critical issues related to air quality, climate change, and ozone depletion.

### PRIOR ARTS

### Patent Document

(Patent Document 1) Korea Patent Registration No. 10-2507977 (METHOD AND SYSTEM RELATED TO RADIOSONDE)

### SUMMARY

Therefore, the present disclosure has been devised to solve the above problems, and an object of the present disclosure is to provide an upper atmospheric environmental gas measurement device for a radiosonde that may be used in a radiosonde by integrating an advanced gas sensor and manufacturing the device in a compact and inexpensive manner, and a measurement method thereof.

However, objects of the present disclosure are not limited to the above-described objects, and other objects not mentioned may be clearly understood by a person having ordinary skill in the technical field to which the present disclosure belongs from the description below.

In order to achieve the objects, according to an aspect of the present disclosure, there is provided an upper atmospheric environmental gas measurement device (100) for a radiosonde (300), including: an insulation pack having an internal space (160); an air intake port (170) whose one end is exposed to an outside of the insulation pack to allow external air to flow in and whose other end extends to the internal space (160); a heater (176) connected to the other end of the air intake port (170) to heat the external air to room temperature; an environmental gas sensor (190) for measuring a concentration of an environmental gas contained in the heated external air; a temperature sensor (210) for measuring temperature of the external air passing through the environmental gas sensor (190); a pump inlet pipe (220) whose one end is connected to the environmental gas sensor (190) and whose middle region passes through the temperature sensor (210); an air pump (230) connected to the other end of the pump inlet pipe (220) and having an outlet (235) exposed to the internal space (160); a control unit (200) controlling the heater (176) based on output of the temperature sensor (210); an X-data communication module (260) transmitting an output signal of the environmental gas sensor (190) to the outside; and a battery (180) supplying power to the upper atmospheric environmental gas measurement device (100).

Optionally, the insulation pack includes an internal insulation pack (130) in which the upper atmospheric environmental gas measurement device (100) is accommodated inside, and an internal insulation cap (140) that forms one surface of the internal insulation pack (130) and is assembled so as to be openable/closable.

Optionally, the upper atmospheric environmental gas measurement device further includes an external insulation pack (110) in which the internal insulation pack (130) is assembled inside; and an external insulation cap (120) that forms one surface of the external insulation pack (110) and is assembled so as to be openable/closable.

Optionally, a hot pack (150) is further provided between the internal insulation cap (140) and the external insulation cap (120).

Optionally, the upper atmospheric environmental gas measurement device for a radiosonde further includes a heater insulation pack (172) surrounding the heater (176) within the internal space (160).

Optionally, the room temperature is in a range of 20° C. to 24° C.

Optionally, the environmental gas is at least one of O₃, NO₂, and SO₂.

Optionally, the environmental gas sensor (190) is a solid polymer electrolyte type gas sensor.

Optionally, the insulation pack includes Styrofoam.

In order to achieve the objects of the present disclosure, according to another aspect, there is provided a radiosonde (300) including: a first upper atmospheric environmental gas measurement device (100a); and at least one second upper atmospheric environmental gas measurement device (100b) connected in series with the first upper atmospheric environmental gas measurement device (100a) via an X-data communication line (250).

In order to achieve the objects of the present disclosure, according to still another aspect, there is provided a measurement method using the above-described upper atmospheric environmental gas measurement device (100), the measurement method including: a step (S100) of sucking sub-zero (for example, −70° C. to −20° C.) upper atmospheric external air through an air intake port (170); a step (S120) of heating the external air to room temperature by a heater (176); a step (S140) of measuring concentration of an environmental gas contained in the external air by an environmental gas sensor (190); a step (S150) of transmitting an output signal of the environmental gas sensor (190) to an outside through an X-data communication module (260) by a control unit (200); a step (S160) of measuring temperature of air discharged from the environmental gas sensor (190) by a temperature sensor (210); a step (S200, S210) of controlling power of the heater (176) based on the measured temperature by the control unit (200); a step (S220) of discharging the air that has passed through the temperature sensor (210) into an internal space (160) by an air pump (230); and a step (S240) of heating the internal space (160) by the air, and then discharging the air to the outside through the internal insulation pack (130) and the external insulation pack (110).

Optionally, the step (S200, S210) of controlling the power of the heater (176) includes a step (S200) of supplying additional power to the heater (176) by the control unit (200) when the measured temperature is equal to or less than a reference value, and a step (S210) of blocking or reducing the power supplied to the heater (176) by the control unit (200) when the measured temperature exceeds the reference value.

According to one embodiment of the present disclosure, compared to an ozone sensor using a conventional electrochemical concentration cell (ECC), a solid electrolyte type sensor is used, so that the manufacturing cost is very low and it is ultra-light. In addition, since the preprocessing process before launching the radiosonde is simple and it can be attached to a commercial radiosonde with just a simple operation, economical and efficient upper atmospheric environmental gas measurement is possible.

In addition, according to the present disclosure, since the measurement device is very light, the size of the balloon used for launching the radiosonde can be reduced, thereby reducing the amount of helium consumed.

And, according to the present disclosure, since a plurality of environmental gas measurement devices can be mounted by launching the radiosonde once, there is an advantage in that multiple environmental gases can be measured simultaneously. For example, when n radiosondes are launched simultaneously, up to 2n environmental gases can be measured simultaneously.

However, effects obtainable from the present disclosure are not limited to the effects mentioned above, and other effects not mentioned will be clearly understood by those skilled in the art to which the present disclosure belongs from the description below.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The following drawings attached to the present specification illustrate preferred embodiments of the present disclosure and, together with the detailed description of the present disclosure described below, serve to further understand the technical idea of the present disclosure, and therefore, the present disclosure should not be interpreted as being limited to matters described in such drawings. The above and other aspects, features and other advantages of the present disclosure will be more clearly understood from the following detailed description taken in conjunction with the accompanying drawings, in which:

FIG. 1 is an internal configuration diagram of an upper atmospheric environmental gas measurement device 100 for a radiosonde according to a first embodiment of the present disclosure;

FIG. 2 is a schematic block diagram of the upper atmospheric environmental gas measurement device 100 illustrated in FIG. 1;

FIG. 3 is a configuration diagram of a radiosonde 300 in which a plurality of upper atmospheric environmental gas measurement devices 100 is configured in series according to a second embodiment of the present disclosure; and

FIG. 4 is a flow chart schematically illustrating an upper atmospheric environmental gas measurement method for a radiosonde according to the present disclosure.

### DETAILED DESCRIPTION OF THE EMBODIMENT

Hereinafter, the exemplary embodiment of the present disclosure will be described with reference to the accompanying drawings and exemplary embodiments as follows. Scales of components illustrated in the accompanying drawings are different from the real scales for the purpose of description, so that the scales are not limited to those illustrated in the drawings.

Hereinafter, with reference to the attached drawings, embodiments of the present disclosure will be described in detail so that a person having ordinary skill in the art to which the present disclosure pertains may easily implement the present disclosure. However, since the description of the present disclosure is merely an embodiment for structural and functional explanation, the scope of the rights of the present disclosure should not be construed as being limited by the embodiments described in the text. That is, since the embodiments may be variously modified and may have various forms, the scope of the rights of the present disclosure should be understood to include equivalents that may realize the technical idea. In addition, the purpose or effect presented in the present disclosure does not mean that a specific embodiment must include all of them or only such effects, and therefore the scope of the rights of the present disclosure should not be understood as being limited thereby.

The meanings of terms described in the present disclosure should be understood as follows.

The terms “first”, “second”, and the like are intended to distinguish one component from another, and the scope of the right should not be limited by these terms. For example, the first component may be referred to as the second component, and similarly, the second component may also be referred to as the first component. When a component is referred to as being “connected” to another component, it should be understood that it may be directly connected to the other component, but there may also be another component in between. On the other hand, when a component is referred to as being “directly connected” to another component, it should be understood that there is no other component in between. Meanwhile, other expressions that describe the relationship between components, such as “between” and “directly between” or “adjacent to” and “directly adjacent to”, should be interpreted in the same way.

A singular expression should be understood to include the plural expression unless the context clearly indicates otherwise, and the terms “comprises” or “have” should be understood to specify the presence of a stated feature, number, step, operation, component, part, or combination thereof, but not to exclude the possibility of the presence or addition of one or more other features, numbers, steps, operations, components, parts, or combinations thereof.

All terms used herein, unless otherwise defined, have the same meaning as commonly understood by a person of ordinary skill in the art to which the present disclosure belongs. Terms defined in commonly used dictionaries should be interpreted as having a meaning consistent with the contextual meaning of the relevant art, and shall not be interpreted as having an ideal or overly formal meaning unless explicitly defined in the present disclosure.

### Configuration of First Embodiment

Hereinafter, a configuration of a preferred first embodiment will be described in detail with reference to the attached drawings.

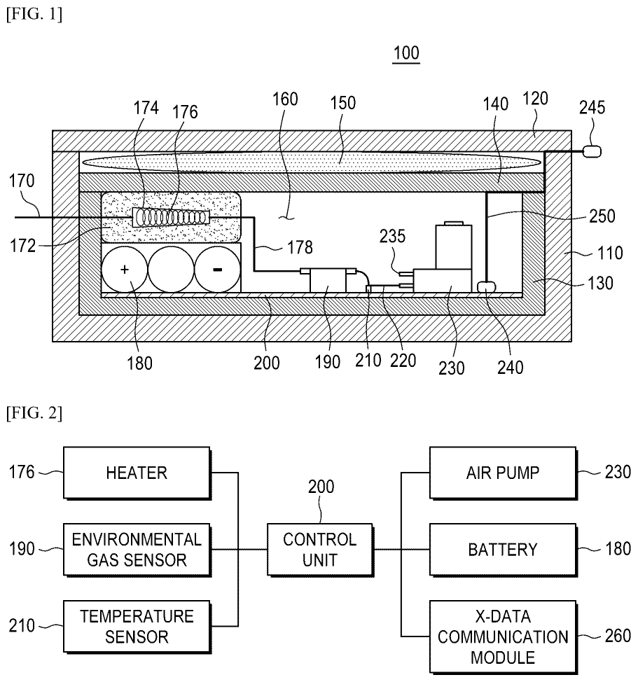

FIG. 1 is an internal configuration diagram of an upper atmospheric environmental gas measurement device 100 for a radiosonde according to the first embodiment of the present disclosure, and FIG. 2 is a schematic block diagram of the upper atmospheric environmental gas measurement device 100 illustrated in FIG. 1. As illustrated in FIGS. 1 and 2, an insulation pack has a rectangular solid shape and an internal space 160. This insulation pack includes an external insulation pack 110, an external insulation cap 120, an internal insulation pack 130, and an internal insulation cap 140.

The internal insulation pack 130 and the internal insulation cap 140 are formed as a rectangular solid with an internal space 160 defined, and the internal insulation cap 140 forms the upper surface of the internal insulation pack 130 and is assembled so as to be openable and closable. The internal insulation pack 130 and the internal insulation cap 140 are made of an insulating material such as Styrofoam and function to keep the internal temperature constant while preventing cold air from the outside from entering.

The external insulation pack 110 and the external insulation cap 120 accommodate the internal insulation pack 130 and the internal insulation cap 140 inside and form a rectangular solid. The external insulation cap 120 forms the upper surface of the external insulation pack 110 and is assembled so as to be openable and closable. The external insulation pack 110 and the external insulation cap 120 are made of an insulating material such as Styrofoam and function to maintain a constant internal temperature while preventing cold air from entering from the outside.

Moreover, a space is formed between the internal insulation cap 140 and the external insulation cap 120, and a hot pack 150 is further provided. This hot pack 150 has the function of uniformly heating the entire upper atmospheric environmental gas measurement device 100. The external insulation cap 120 is opened before launching the radiosonde 300, the hot pack 150 is placed, and then the external insulation cap 120 is closed. Optionally, the hot pack 150 may be omitted.

An air intake port 170 is a thin and long non-metallic tube, one end of which is exposed to the outside of the external insulation pack 110, a middle region passes through the external insulation pack 110 and the internal insulation pack 130, and the other end is connected to a heater capsule 174. Cold air (for example, −70° C. to −20° C.) from the outside is introduced through the air intake port 170.

One end of the heater capsule 174 is connected to the air intake port 170, the other end is connected to a heated air tube 178, and a heater 176 is provided inside the heater capsule 174. The heater 176 uses a heater coated with Tetron to heat the incoming cold air to room temperature (for example, 20° C. to 24° C.). In addition, a heater insulation pack 172 surrounding the heater 176 is further included to improve the heating performance of the heater 176. The heater insulation pack 172 may be made of Styrofoam, or the like.

One end of the heated air tube 178 is connected to the heater capsule 174, and the other end is connected to an environmental gas sensor 190.

The environmental gas sensor 190 is a solid polymer electrolyte type gas sensor and measures the concentration of environmental gases (for example, O₃, NO₂, SO₂, or the like) contained in the external air. The environmental gas sensor 190 is installed in the internal space 160. Compared to the conventional liquid electrolyte type, the solid polymer electrolyte type gas sensor is small (for example, 10×10×10mm 3 ), lightweight (for example, 5 to 10 g), and inexpensive (for example, $40 to $60). In addition, the environmental gas sensor 190 of the present embodiment is for ground use and therefore does not operate properly in extremely low temperature air (for example, −70° C. to −20° C.). Therefore, the sensor is preheated to room temperature using the heater 176 before measurement. In addition, the internal space 160 is also maintained at a constant temperature through an insulating material so that the environmental gas sensor 190 may operate normally.

One end of the pump inlet pipe 220 is installed at the outlet of the environmental gas sensor 190, the middle region passes through the temperature sensor 210, and the other end is connected to the inlet of the air pump 230.

The temperature sensor 210 measures the temperature of the external air passing through the environmental gas sensor 190 and transmits the measured temperature to the control unit 200.

The air pump 230 is installed in the internal space 160, an inlet thereof is connected to the pump inlet pipe 220, and an outlet 235 is exposed to the internal space 160. Since the discharged air is room temperature air, rather than being discharged into the atmosphere as it is, the air passes through the internal space 160 and heats the internal space 160, and then discharged into the atmosphere through the gap between the internal insulation cap 140 and the external insulation cap 120. The air pump 230 may be replaced with a vacuum pump, a compressor, or the like.

The control unit 200 may be a printed circuit board (PCB) provided on the inner bottom surface of the internal insulation pack 130, and may be implemented with a central processing unit (CPU), microcomputer (MICOM), application processor (AP), programmable logic controller (PLC), or the like.

An X-data communication module 260 transmits the output signal of the environmental gas sensor 190 to the outside. To this end, the X-data communication module 260 includes first and second X-data connectors 240 and 245 and an X-data communication line 250 connecting them. In particular, the first X-data connector 240 may be mounted on the control unit 200, and the second X-data connector 245 may be exposed to the outside through an external insulation pack 110. In addition, the second X-data connector 245 includes an X-data IN terminal and an X-data OUT terminal.

A battery 180 is installed in the internal space 160 and supplies power to the upper atmospheric environmental gas measurement device 100.

### Operation of First Embodiment

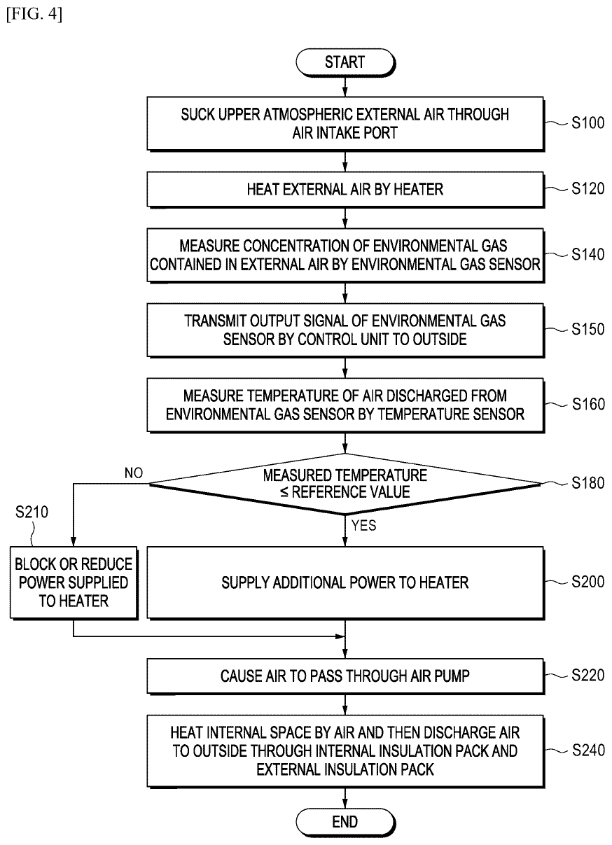

FIG. 4 is a flow chart schematically illustrating a method for measuring upper atmospheric environmental gases for a radiosonde according to the present disclosure. As illustrated in FIG. 4, first, as the air pump 230 operates, upper atmospheric external air at a temperature below zero (for example, −70° C. to −20° C.) is sucked in through the air intake port 170 (S100).

Next, the heater 176 heats the external air to room temperature (for example, 20° C. to 24° C.) (S120). In the present embodiment, the heater 176 heats the external air to a temperature within the range of 20±5° C.

Next, the environmental gas sensor 190 measures the concentration of environmental gas contained in the external air (S140). Then, the measured concentration is transmitted to the control unit 200 and stored.

Next, the control unit 200 transmits the output signal of the environmental gas sensor 190 to the outside through the X-data communication module 260 (S150). Accordingly, a worker on the ground may receive the concentration of the environmental gas in real time through X-data communication.

Next, the temperature sensor 210 measures the temperature of the air discharged from the environmental gas sensor 190 (S160). The measured temperature is transmitted to the control unit 200.

The control unit 200 controls the power of the heater 176 based on the measured temperature (S200 and S210). More specifically, if the measured temperature is equal to or less than a reference value (for example, 20° C.), the control unit 200 supplies additional power to the heater 176 to heat the external air more hotly (S200). When the measured temperature exceeds the reference value (for example, 22° C.), the control unit 200 blocks or reduces the power supplied to the heater 176 to heat or cool the external air less hotly (S210).

To this end, the control unit 200 may select algorithms such as on/off control, proportional (P) control, proportional differential (PD) control, proportional integral (PI) control, and proportional differential integral (PID) control.

Next, the air pump 230 discharges the air that has passed through the temperature sensor 210 into the internal space 160 (S220). Accordingly, the internal space 160 is heated close to room temperature, and waste heat may be further utilized to maintain heat. In addition, by maintaining the temperature of the internal space 160, malfunctions of the control unit 200 and sensors may be reduced.

Then, the air heats the internal space 160 and is discharged to the outside through the internal insulation pack 130 and the external insulation pack 110 (S240).

### Second Embodiment

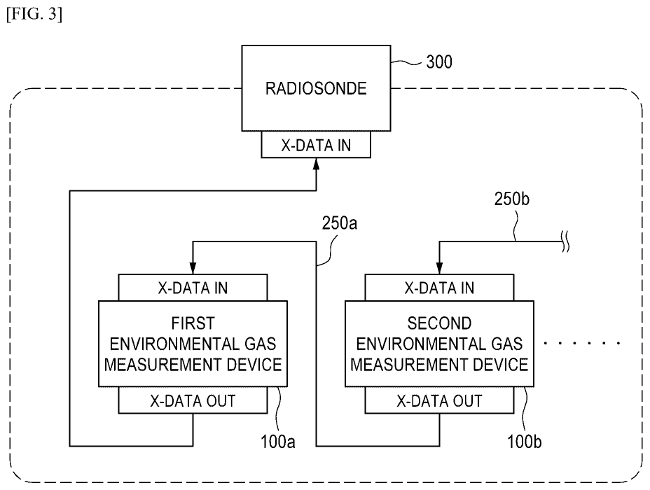

Hereinafter, a second embodiment will be described in detail with reference to the attached drawings. FIG. 3 is a configuration diagram of a radiosonde 300 in which a plurality of upper atmospheric environmental gas measurement devices 100 are configured in series according to the second embodiment of the present disclosure. As illustrated in FIG. 3, a plurality of environmental gas measurement devices is connected in series to the radiosonde 300. For example, in FIG. 3, a first environmental gas measurement device 100a is connected in series between the radiosonde 300 and a second environmental gas measurement device 100b via an X-data communication line 250a. That is, one end of the X-data communication line 250a is connected to the X-data IN of the first environmental gas measurement device 100a, and the other end of the X-data communication line 250a is connected to the X-data OUT of the second environmental gas measurement device 100b. By repeating this serial connection, a plurality of environmental gas measurement devices 100a, 100b, and 100c, . . . is provided.

In this case, the first environmental gas measurement device 100a is dedicated to measuring the concentration of ozone, and the second environmental gas measurement device 100b is dedicated to measuring the concentration of SO₂.

The detailed description of the preferred embodiments of the present disclosure disclosed above has been provided to enable those skilled in the art to implement and practice the present disclosure. While the above has been described with reference to preferred embodiments of the present disclosure, it will be understood by those skilled in the art that various modifications and changes may be made to the present disclosure without departing from the scope of the present disclosure. For example, those skilled in the art may utilize each of the configurations described in the above-described embodiments in a manner that combines them. Accordingly, the present disclosure is not intended to be limited to the embodiments illustrated herein, but is to be accorded the widest scope consistent with the principles and novel features disclosed herein.

The present disclosure may be embodied in other specific forms without departing from the spirit and essential characteristics of the present disclosure. Accordingly, the above detailed description should not be construed in all aspects as restrictive but rather as illustrative. The scope of the present disclosure should be determined by a reasonable interpretation of the appended claims, and all changes coming within the equivalent scope of the present disclosure are intended to be embraced therein. The present disclosure is not intended to be limited to the embodiments set forth herein but is to be accorded the widest scope consistent with the principles and novel features disclosed herein. In addition, claims that do not have an explicit citation relationship in the claims may be combined to constitute an embodiment or may be included as a new claim by post-application amendment.

### DESCRIPTION OF NUMBERS IN DRAWINGS

100: Upper Atmospheric Environmental Gas Measurement Device
100a: First Upper Atmospheric Environmental Gas Measurement Device
100b: Second Upper Atmospheric Environmental Gas Measurement Device
110: External Insulation Pack
120: External Insulation Cap
130: Internal Insulation Pack
140: Internal Insulation Cap
150: Hot Pack
160: Internal Space
170: Air Intake Port
172: Heater Insulation Pack
174: Heater Capsule
176: Heater
178: Heated Air Tube
180: Battery
190: Environmental Gas Sensor
200: Control Unit
210: Temperature Sensor
220: Pump Inlet Pipe
230: Air Pump
235: Outlet
240: First X-data Connector
245: Second X-data Connector
250, 250a, 250b: X-data Communication Line
260: X-data Communication Module
300: Radiosonde

What is claimed is:

1. An upper atmospheric environmental gas measurement device for a radiosonde, comprising:
an insulation pack having an internal space;
an air intake port whose one end is exposed to an outside of the insulation pack to allow external air to flow in and whose other end extends to the internal space;
a heater connected to the other end of the air intake port to heat the external air to room temperature;
an environmental gas sensor for measuring a concentration of an environmental gas contained in the heated external air;
a temperature sensor for measuring temperature of the external air passing through the environmental gas sensor;
a pump inlet pipe whose one end is connected to the environmental gas sensor and whose middle region passes through the temperature sensor;
an air pump connected to the other end of the pump inlet pipe and having an outlet exposed to the internal space;
a control unit controlling the heater based on output of the temperature sensor;
an X-data communication module transmitting an output signal of the environmental gas sensor to the outside; and
a battery supplying power to the upper atmospheric environmental gas measurement device.

2. The upper atmospheric environmental gas measurement device for a radiosonde according to claim 1, wherein the insulation pack includes
an internal insulation pack in which the upper atmospheric environmental gas measurement device is accommodated inside, and
an internal insulation cap that forms one surface of the internal insulation pack and is assembled so as to be openable/closable.

3. The upper atmospheric environmental gas measurement device for a radiosonde according to claim 2, further comprising:
an external insulation pack in which the internal insulation pack is assembled inside; and
an external insulation cap that forms one surface of the external insulation pack and is assembled so as to be openable/closable.

4. The upper atmospheric environmental gas measurement device for a radiosonde according to claim 3, wherein a hot pack is further provided between the internal insulation cap and the external insulation cap.

5. The upper atmospheric environmental gas measurement device for a radiosonde according to claim 1, further comprising a heater insulation pack surrounding the heater within the internal space.

6. The upper atmospheric environmental gas measurement device for a radiosonde according to claim 1, wherein the room temperature is in a range of 20° C. to 24° C.

7. The upper atmospheric environmental gas measurement device for a radiosonde according to claim 1, wherein the environmental gas is at least one of O₃, NO₂, and SO₂.

8. The upper atmospheric environmental gas measurement device for a radiosonde according to claim 1, wherein the environmental gas sensor is a solid polymer electrolyte type gas sensor.

9. The upper atmospheric environmental gas measurement device for a radiosonde according to claim 1, wherein the insulation pack includes Styrofoam.

10. A radiosonde comprising:
a first upper atmospheric environmental gas measurement device according to claim 1; and
at least one second upper atmospheric environmental gas measurement device connected in series with the first upper atmospheric environmental gas measurement device via an X-data communication line.

11. A measurement method using the upper atmospheric environmental gas measurement device according to claim 1, the measurement method comprising:
a step of sucking sub-zero upper atmospheric external air through an air intake port;
a step of heating the external air to room temperature by a heater;
a step of measuring concentration of an environmental gas contained in the external air by an environmental gas sensor;
a step of transmitting an output signal of the environmental gas sensor to an outside through an X-data communication module by a control unit;
a step of measuring temperature of air discharged from the environmental gas sensor by a temperature sensor;
a step of controlling power of the heater based on the measured temperature by the control unit;
a step of discharging the air that has passed through the temperature sensor into an internal space by an air pump; and
a step of heating the internal space by the air, and then discharging the air to the outside through the internal insulation pack and the external insulation pack.

12. The measurement method according to claim 11, wherein the step of controlling the power of the heater includes
a step of supplying additional power to the heater by the control unit when the measured temperature is equal to or less than a reference value, and
a step of blocking or reducing the power supplied to the heater by the control unit when the measured temperature exceeds the reference value.
